(19) United States
(12) Patent Application Publication
(10) Pub. No.: US 2015/0110717 A1
(43) Pub. Date: Apr. 23, 2015

(54) METHODS OF INCREASING THE NUMBER OF TARGET CELLS RECOVERED FROM A FLUID SAMPLE

(71) Applicants: Robert J. Distel, Newton, MA (US); Yvon Cayre, Paris (FR)

(72) Inventors: Robert J. Distel, Newton, MA (US); Yvon Cayre, Paris (FR)

(73) Assignees: SCREENCELL, Paris (FR); DANA FARBER CANCER INSTITUTE, INC., Boston, MA (US)

(21) Appl. No.: 14/342,888

(22) PCT Filed: Sep. 7, 2012
(86) PCT No.: PCT/US2012/054241

§ 371 Date: Nov. 7, 2014

Related U.S. Application Data
(60) Provisional application No. 61/531,884, filed on Sep. 7, 2011.

(51) Int. Cl. A01K 67/027; G01N 33/574; C12N 5/09

(52) U.S. Cl. CPC A01K 67/0271; C12N 5/0694; G01N 33/574; A01K 2227/105; A01K 2267/0331
USPC 424/9.2; 800/10; 435/7.23

(57) ABSTRACT

Methods and materials for increasing the number of target cells recovered from a fluid sample containing cells are described. The methods include isolating the target cells on a filter and then implanting the filter containing the target cells in an immunodeficient non-human animal, where at least some of the target cells can proliferate.

### CROSS-REFERENCE TO RELATED APPLICATIONS

This application claims priority to U.S. Application Ser. No. 61/531,848, filed on Sep. 7, 2011, the disclosure of which is incorporated by reference in its entirety.

### TECHNICAL FIELD

This invention relates to methods and materials for increasing the number of target cells recovered from a fluid sample containing cells, and more particularly to increasing the number of target cells recovered from a fluid sample containing cells by isolating the target cells on a filter and then implanting the filter containing the target cells in an immunodeficient non-human animal, where at least some of the target cells can proliferate.

### BACKGROUND

Despite considerable progress in diagnosing and treating solid tumors, metastatic disease remains the foremost cause of cancer-related death. Although the mechanisms of metastasis development are yet to be fully elucidated, circulation of tumor cells derived from the primary tumor in the bloodstream of a patient is a fundamental intermediate event in the metastatic cascade. Detecting circulating tumor cells (CTCs) is important for patient care for a number of reasons, including earlier detection of secondary tumors, monitoring response to therapy, and monitoring disease progression. However, as there is such a small number of CTCs in the blood (e.g., about 1 CTC per 109 cells in peripheral blood of patients with metastatic cancer), there continues to be a need for methods and material for capturing, detecting, and growing CTCs.

### SUMMARY

This document is based on the discovery of methods for increasing the number of target cells recovered from a fluid sample containing cells. As described herein, target cells can be retained on or in a filter on the basis of size, and then the filter containing the target cells can be implanted into a non-human animal, preferably an immunodeficient non-human animal, where the cells can proliferate. Such methods can be used to increase the number of rare circulating cells such as CTCs recovered from a peripheral blood sample and create individualized animal models of a patient's tumor that can be used, for example, to evaluate the patient's tumor, assess metastatic potential of the cells, and to determine responsiveness of the cells to different chemotherapeutics.

In one aspect, this document features a method of increasing the number of target cells from a fluid sample comprising cells. The method includes providing a filter comprising one or more target cells, the one or more target cells obtained from a fluid, target cell and non-target cell containing sample by passage of the sample through a filtration device comprising the filter, wherein the size of pores in the filter causes the target cells to be retained on or in the filter; and implanting the filter and the one or more target cells on the filter in a non-human animal, e.g., an immunodeficient non-human animal such as an immunodeficient mouse, wherein some or all of the one or more cells on or in the implanted filter proliferate in the animal. During the passage of the sample through the filtration device, substantially all the non-target cells can pass through the filter. The immunodeficient mouse can be homozygous for the severe combined immune deficiency (SCID) spontaneous mutation (Prkdc^{scid}); homozygous for the nude spontaneous mutation (Foxn1^{nu/nu}0); homozygous for a Rag1 mutation; homozygous for a Rag2 mutation; or homozygous for both the Rag1 and the Rag2 mutation.

The method further can include providing one to four additional filters (e.g., one, two, three or four additional filters), each additional filter comprising one or more target cells, and implanting the first and additional filters in the immunodeficient non-human animal. The first and additional filters can be obtained from a single filtration device or from separate filtration devices. The method further can include, before implanting the filter and the one or more target cells on the filter, stacking the filters substantially on top of each other to produce a multi-layered culture device.

Any of the methods described herein further can include, before implanting the filter and the one or more target cells on the filter, contacting the surface of the filter and any additional filters comprising the target cells with a composition that can transition from a liquid to gel phase without lethal or toxic effects on the target cells. The composition can include one or more extracellular matrix (ECM) components (e.g., reconstituted basement membrane).

In any of the methods described herein, the filter can include one or more compounds immobilized thereto. In any of the methods described herein, one or more compounds can be administered to the immunodeficient animal. For example, the one or more compounds can be selected from the group consisting of a growth factor, an extracellular matrix protein, an enzyme, a reporter molecule, a liposome, and a nucleic acid. The growth factor can be epidermal growth factor (EGF), platelet derived growth factor (PDGF), keratinocyte growth factor (KGF), a fibroblast growth factor (FGF), or a transforming growth factor (TGF). The extracellular matrix protein can be collagen, laminin, fibronectin, or heparan sulfate. The reporter molecule can include a fluorophore-quencher dual labeled probe that is a substrate for a metalloproteinase.

In any of the methods described herein, the method further can include monitoring growth of the cells in the immunodeficient animal.

In any of the methods described herein, the fluid, cell-containing sample can include peripheral blood cells or can include cells from urine, bone marrow, lymph, lymph node, spleen, cerebral spinal fluid, ductal fluid, a biopsy specimen, or a needle biopsy aspirate.

In any of the methods described herein, the method further can include, before the implanting step, culturing the one or more target cells.

In any of the methods described herein, the target cells can be cancer cells, circulating cancer cells, fetal cells, or stem cells (e.g., endothelial stem cells or mesenchymal stem cells).

In another aspect, this document features a non-human immunodeficient animal that includes at least one implanted filter, the filter comprising a plurality of target cells obtained from a fluid, target cell and non-target cell containing sample by passage through a filtration device comprising the filter, wherein the size of pores in the filter causes the target cells to be retained on or in the filter. The animal further can include one to four additional implanted filters (e.g., one, two, three or four), each said additional filter comprising one or more target cells. The first and additional filters can be obtained from a single filtration device or from separate filtration devices. The first and additional filters can be substantially stacked on top of each other to produce a multi-layered three-dimensional culture device. The surface of the filter and any additional filters comprising the target cells can include a composition that can transition from a liquid to gel phase without lethal or toxic effects on the target cells (e.g., human target cells).

This document also features a method of testing for the presence of tumor cells in fluid sample comprising test cells. The method includes providing a filter comprising a plurality of test cells obtained from a fluid, test cell-containing sample by passage through a filtration device comprising the filter, wherein the size of pores in the filter causes one or more of the test cells to be retained on or in the filter; implanting the filter and the one or more of the test cells on or in the filter in the immunodeficient non-human animal; and monitoring the immunodeficient non-human animal for the presence or absence of a tumor, wherein the presence of a tumor indicates that the test cells comprised tumor cells. The test cells can be human cells. The fluid, cell-containing sample can include peripheral blood cells or comprise cells from urine, bone marrow, lymph, lymph node, spleen, cerebral spinal fluid, ductal fluid, a biopsy specimen, or a needle biopsy aspirate. The method further can include administering a chemotherapeutic agent to the non-human animal if the tumor is present; and monitoring the tumor for responsiveness to the chemotherapeutic agent.

In another aspect, this document features a method of increasing the number of target cells from a fluid sample comprising cells. The method includes providing a fluid, target cell- and non-target cell-containing sample; passing the sample through a filtration device, the device comprising a filter support fastened to a filter, a compartment having an upper opening and a lower opening, and means mobile relative to the compartment for applying a force to the support and releasing the support; removing, from the device, the filter containing one or more target cells; and implanting the filter and the target cells on or in the filter into the immunodeficient non-human animal, wherein some or all of the one or more target cells on or in the implanted filter proliferate in the immunodeficient animal.

Unless otherwise defined, all technical and scientific terms used herein have the same meaning as commonly understood by one of ordinary skill in the art to which this invention belongs. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a perspective view of a ScreenCell® filtration device for recovering target cells from a fluid sample according to an embodiment described herein.

FIGS. 2A and 2C are perpendicular axial sections of the embodiment of FIG. 1, with the parts illustrated in FIG. 1 assembled. The circled portions in FIGS. 2A and 2C are presented in enlarged view in FIGS. 2B and 2D, respectively.

FIGS. 3A-3O are elevation or cross-section views of the ScreenCell® filtration device of FIG. 1 from storage configuration (3A) through each step of using the device (3B-3O) for isolating and recovering target cells from a fluid sample.

FIG. 4 is perspective view of one embodiment of a ScreenCell® filtration device, before use.

FIG. 5 diagrammatically represents the embodiment of the ScreenCell® device illustrated in FIG. 4 after removal of a protective film and before insertion of a vacuum tube.

FIG. 6 diagrammatically represents the embodiment of the ScreenCell® device illustrated in FIGS. 4 and 5, after insertion of the vacuum tube,

FIG. 7 diagrammatically represents the embodiment of the ScreenCell® device illustrated in FIGS. 4-6 during removal of the vacuum tube and a protective cylinder.

FIG. 8A is a photomicrograph of live H2030 cells following filtration through a ScreenCell® Cyto device. FIG. 8B is a photomicrograph of the cells from FIG. 8A after culturing the filter for 4 days in culture medium. Cells in FIGS. 8A and 8B are representative of 8 independent experiments; cells were observed under a microscope (×40).

Like reference symbols in the various drawings indicate like elements.

### DETAILED DESCRIPTION

In general, this document is based on methods and materials for increasing the number of target cells recovered from a fluid sample that contains target and non-target cells. The term “fluid sample containing cells” refers to a liquid containing a suspension of cells. Non-limiting examples include biological fluids such as blood (e.g., peripheral blood or umbilical cord blood), urine, lymph, cerebral spinal fluid, or ductal fluid, or such fluids diluted in a physiological solution (e.g., saline, phosphate-buffered saline (PBS), or tissue culture medium), or cells obtained from biological fluids (e.g., by centrifugation) and suspended in a physiological solution. Other examples of a “fluid sample containing cells” include cell suspensions (in physiological solutions) obtained from bone marrow aspirates, needle biopsy aspirates or biopsy specimens from, for example, lymph node or spleen. Such fluid samples can be obtained from any mammalian subject, including humans, monkeys, mice, rats, rabbits, guinea pigs, dogs, or cats. Fluid samples from human subjects are particularly useful. In embodiments in which the fluid sample contains red blood cells, the red blood cells can be selectively lysed using, for example, a buffer containing ammonium chloride or saponin or removed by, for example, density gradient sedimentation or hetastarch aggregation.

As described herein, viable target cells can be recovered from a fluid sample on or in a filter, which then can be implanted in an immunodeficient non-human animal, where the target cells can proliferate. Target cells can include fetal blood cells, circulating tumor cells (CTCs), disseminated tumor cells (DTCs) (i.e., tumor cells in bone marrow), or stem cells (e.g., cancer stem cells, mesenchymal stem cells, or endothelial stem cells). For example, fetal blood cells can be recovered from a sample of maternal blood (optionally diluted in a physiological solution) and used for non-invasive prenatal diagnosis. One or more filters containing target cells recovered from a patient having, or suspected of having, a cancer (e.g., breast, ovarian, colon, lung, pancreatic, kidney, liver, prostate, melanoma, bladder, thyroid, or lymphoma) can be implanted in the immunodeficient non-human mammal to increase the number of target cells (including the progeny of target cells trapped in or on a filter) for further characterization, including one or more of genomic, proteomic, immunocytochemistry, or fluorescence in situ hybridization (FISH) assays, to, for example, aid in prognosis determination. Implantation into an immunodeficient animal also allows a determination of the capability of the target cells to initiate tumors and metastasize and/or a determination of the responsiveness of the cell to one or more chemotherapeutic agents.

Non-target cells are all the cells in a fluid sample containing cells other than the target cells. Thus, for example, in blood in which the target cells are CTCs, non-target cells would include red blood cells, lymphocytes (T and B), monocytes, and granulocytes, which are smaller than most cancer cells. Where the fluid sample containing cells is, for example, a cell suspension prepared from lymph node tissue and the target cells are cancer cells, non-target cells will include lymphocytes (T and B), monocytes, macrophages, and granulocytes, which are smaller than most cancer cells.

### Filtration Device

A fluid sample containing target and non-target cells can be passed through a filtration device that includes a filter, wherein the size of the pores in the filter causes the target cells to be retained on or in the filter. To prepare the sample for passage through the filtration device, the sample is typically diluted with a buffer containing culture medium (e.g., RPMI such as RPMI 1640, DMEM, or MEM) supplemented with bovine serum albumin, a red blood cell lysis agent such as ammonium chloride, saponin, or potassium bicarbonate, a biocidal agent such as sodium azide or a hypochlorite solution (0.1 to 2 mM), and optionally a calcium channel blocker such as amlodipine, benidipine, or barnidipine, and incubated for one to five minutes (e.g., one minute, two minutes, three minutes, four minutes, or five minutes). For example, the buffer can be supplemented with 0.2 to 2 g (e.g., 0.2, 0.3, 0.4, 0.5, 0.6, 0.7, 0.8. 0.9, 1.0, 1.2, 1.4, 1.6, 1.8, or 2.0 g) of BSA, 0.01 to 0.1 g (e.g., 0.01, 0.02, 0.03, 0.04, 0.05, 0.06, 0.07, 0.08, 0.09, or 1.0 g) of a red blood cell lysis agent, 0.1 to 2 mM (e.g., 0.2, 0.3, 0.4, 0.5, 0.6, 0.7, 0.8. 0.9, 1.0, 1.2, 1.4, 1.6, 1.8, or 2.0 mM) of a biocidal agent, and 5 to 30 nM (e.g., 5, 10, 15, 20, 25, or 30 nM) of a calcium channel blocker. After incubating, a cell culture medium (e.g., RPMI) is added to the diluted sample, which then is filtered through the device. During the passage of the sample through the filtration device, substantially all, i.e., greater than 99%, of the non-target cells pass through the filter. The non-target cells of the fluid sample can pass through the filter by the application of a vacuum to the underside of the filter. In some embodiments, the non-target cells of the sample are caused to pass through the filter by introducing a tube inside the holder of the device which, once its rubber cap is pierced, establishes a pressure difference between the blood volume and the vacuum in the tube, forcing blood through the filter and into the tube.

The filtration device containing the filter is not limited to a particular structure and can be of any shape, size, or material as long as it is a) capable of receiving the fluid sample containing the cells and supporting a filter in which the size of its pores retains the target cells on or in the filter, and b) is configured for removal of the fluid after passage of the sample through the filter. For example, a filtration device can be composed of one or more of the following: a plastic made of one or more polymers such as polycarbonate, polyamide, polyvinyl chloride, polypropylene, polyethylene, or a polyetheretherketone such as PEEK™; a metal alloy such as stainless steel (e.g., surgical steel); a ceramic, glass; or a composite material. The filtration devices of ScreenCell® (Paris, France) are particularly useful and have been used in the methods described herein. See also the filtration devices described in U.S. Patent Publication No. 20110070642, U.S. Patent Publication No. 20110104670, U.S. Patent Publication No. 20090081772, U.S. Provisional Application No. 61/417,526, WO2011055091, WO2009106760, and WO2009047436, each of which is incorporated by reference in its entirety.

In one embodiment, the filtration device includes a compartment for receiving a fluid sample and a filter mounted, at least temporarily, onto an opening of the compartment. The filtration device further can include a needle mounted, at least temporarily, onto the opening of the compartment. In such an embodiment, the filter is located between the needle and the interior capacity of the compartment. The mounted needle is designed to pierce the plug of a vacuum tube (e.g., a blood vacuum tube), creating negative pressure relative to ambient pressure in order to aspirate the liquid through the filter. Such a filtration device allows viable cells to be isolated and collected on a filter under conditions compatible with further culturing and/or implantation into an immunodeficient non-human animal.

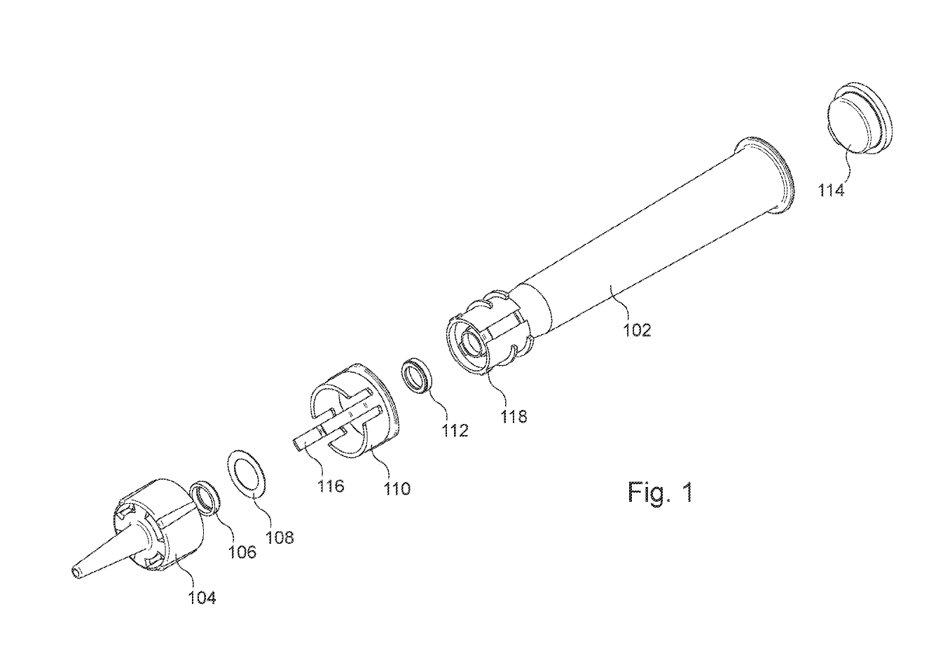

FIG. 1 shows one embodiment of a filtration device (ScreenCell®, Paris—France) that includes a reservoir or compartment, 102, an end-piece 104, a seal 106, a filter with its support 108, a movable means 110, a seal 112 and a plug 114. The compartment 102 is substantially cylindrically shaped. Its upper end can be sealed by the plug 114 in such a way that it is impermeable. The lower end of the compartment 102 has, on its external surface, discontinuous rings with gaps which guide legs on the movable means 110, said rings guiding the body of the movable means 110.

This movable means 110 is generally cylindrical in shape and is supplied with two legs extending towards the end-piece 104 and narrowing together in this direction so that there is a separation between them measuring less than the diameter of the filter support 108. As will be subsequently illustrated, this particular shape, notably that of the legs 116 curved towards each other, allows the movable means 110, after it is removed from the end-piece 104, to apply pressure to the filter support 108 so that the filter support is released from the compartment 102, along with its filter, while the movable means is being advanced towards the filter support 108.

The ends of the legs 116 of the movable means 110 and the lower end of the compartment 102 are designed to be inserted into a cell culture box or well. The diameter of the discontinuous ring at the end of the compartment 102 is designed so that the compartment can be supported on the edge of a cell culture box or well.

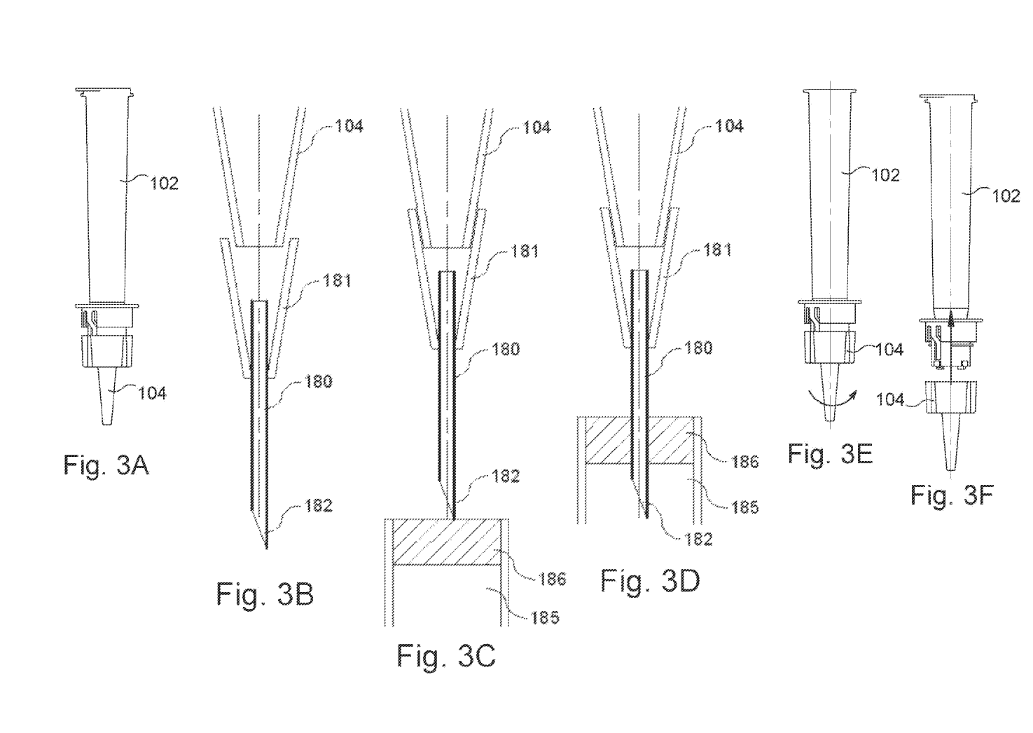

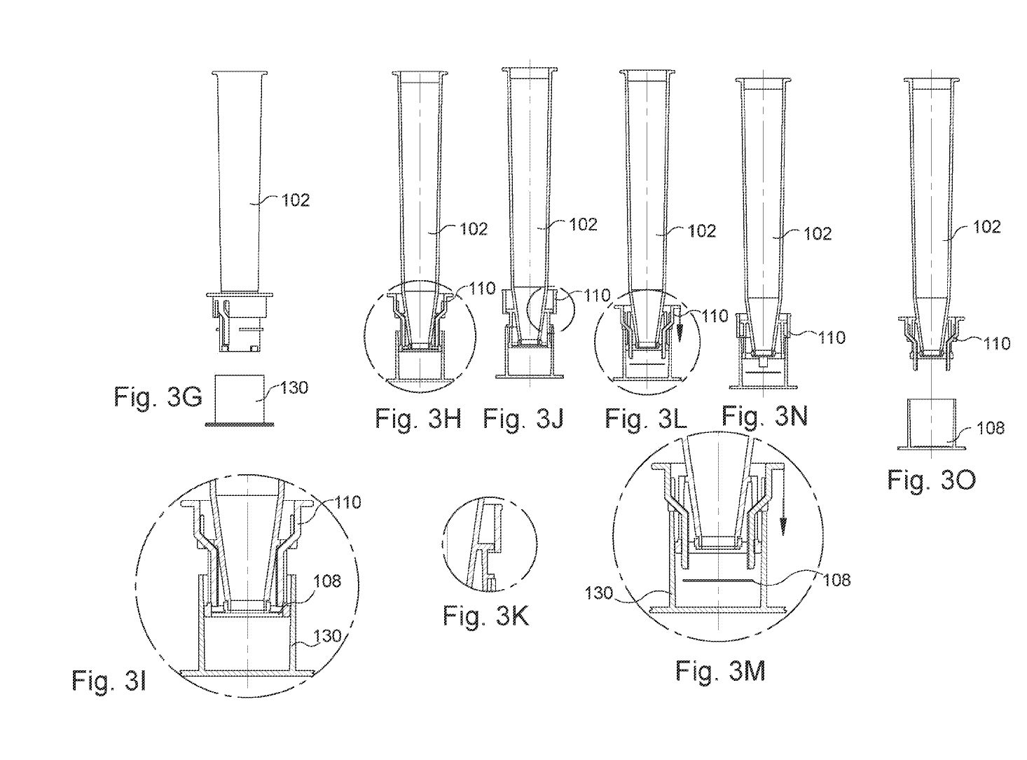

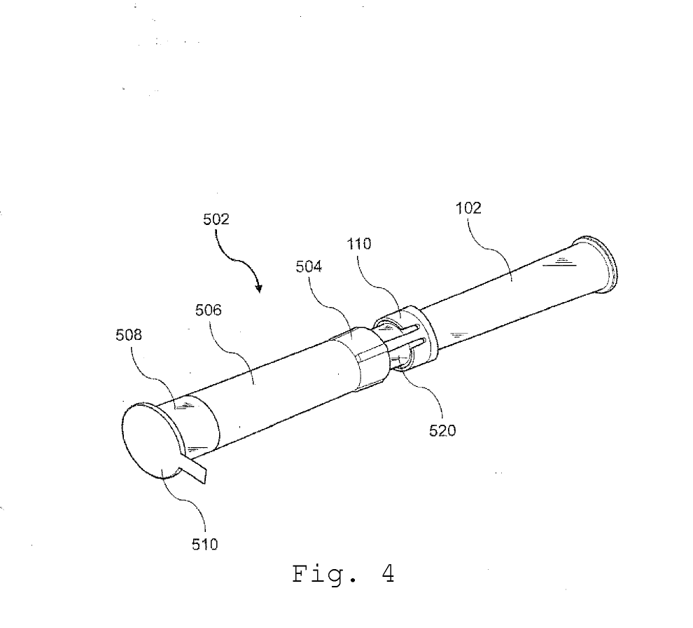

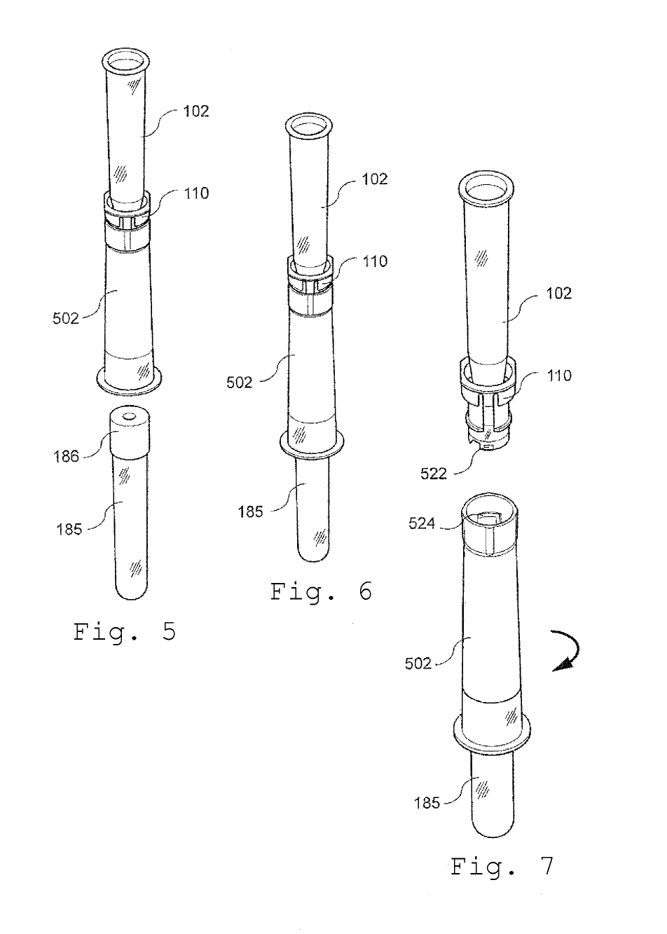

An end-piece or adaptor 104, is located at the lower opening of the compartment 102. End piece or adaptor 104 grips the exterior wall of the compartment 102 and has a lower narrow opening smaller in diameter than that of the compartment 102. End-piece or adaptor 104 is detachable, impermeable, and sterile. The lower narrow opening in the end-piece or adaptor 104 is sufficiently long to enable a leak-free mechanical fit of the aperture of a needle (see FIGS. 3B to 3D).

In a manner coordinated with the shape of the lower end of the compartment 102, which has lateral lugs 118, the end-piece 104 has rotation locking means for gripping the lugs in the manner known. In this way, the end-piece 104 allows the filter-holder to be held in place during filtration. In addition, the end-piece 104 protects the filter from splashes and any potential contamination.

When attached, the lower end of the compartment 102 has an opening which discharges onto the filter held by the filter support 108, which is itself held in position, on the one hand, by the lower end of the compartment 102 and, on the other, by the end-piece 104.

In one embodiment, the filter support 108 is shaped as a ring-like disk. The filter is micro-perforated and is fused to the underside of the filter support 108, then inserted along with it into the lower end of the compartment 102.

The filter support 108 can be ring-shaped and made, for example, of plastic such as polyvinyl chloride (PVC) or a metal alloy such as surgical steel. A filter support made of surgical steel is particular useful in the filtration device. The thickness of the ring is designed to allow it to be scanned. The filter support can include an identifier such that the collected cells can be associated with a patient. In one embodiment, the external diameter of the filter support can be, for example, 12 to 13 mm such as 12.6 mm and the diameter of the filter to which the filter support 108 is attached can be 5.5 to 6.5 mm such as 5.9 mm.

In one embodiment, the compartment 102, the end-piece 104 and the movable means 110 are composed of polypropylene, for example. The seals 106 and 112 are made of silicone, for example.

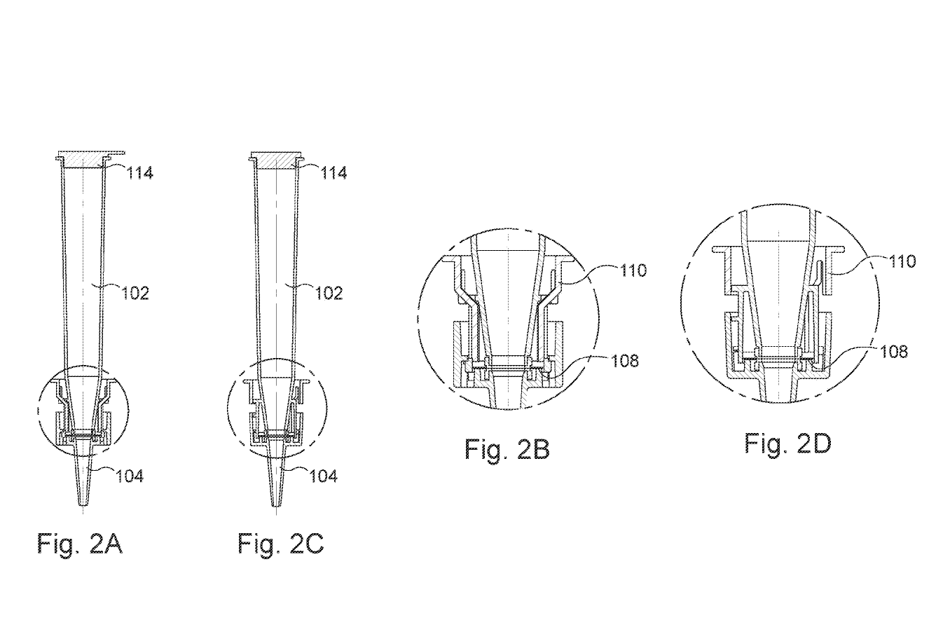

FIGS. 2A and 2C are views of perpendicular axial sections of the embodiment depicted in FIG. 1. FIGS. 2B and 2D are enlarged views of the circled portions of FIGS. 2A and 2C, respectively.

FIG. 3A represents the ScreenCell® filtration device in elevation, in its storage configuration. FIG. 3B represents the insertion of the end-piece 104 into the aperture 181 of a needle 180, which has another very fine, beveled end 182 to make it easier to pierce a vacuum tube plug. The aperture 181 of the needle 180 is preferentially made of plastic. The end 182 of the needle 180 is preferentially metallic. The needle 180 can be positioned on the end-piece 104 either before or after liquid (not shown), for example blood, is introduced into the compartment 102, through its upper opening.

FIG. 3C illustrates the vacuum tube 185 plug 186 beginning to be pierced, once the needle 180 is impermeably joined to the end-piece 104.

FIG. 3D illustrates the plug 186 completely pierced through by the needle 180, connecting the interior of the negative pressure vacuum tube 185, through the filter 108, to the volume of the compartment 102 holding the liquid containing the cells of interest. The inner volume of the vacuum tube 185 is greater than the volume of the liquid to be filtered.

During filtration, some target cells in the fluid sample present in the compartment 102, which are larger in diameter, are retained by the filter 108 while substantially all of the liquid contents and the cells smaller in dimension than the target cells are aspirated into the vacuum tube 185, through the filter 108.

Next, as illustrated in FIGS. 3E and 3F, the end-piece 104 is removed after being rotated to release it from the lugs 118. Then, as illustrated in FIGS. 3G and 3H, the end of the compartment 102 is inserted into a cell culture box or well 130.

As explained above and illustrated in FIG. 31, the end near to the legs 116 of the movable means 110 and the lower end of the compartment 102 are designed to be inserted into a cell culture box or well. In contrast, the discontinuous ring at the end of the compartment 102 has a diameter allowing it to be supported on the edge of the culture box or well 130.

To be more precise, as illustrated in FIGS. 3J and 3K, the movable means 110 can in this position still move parallel to the axis of the compartment 102.

As illustrated in FIGS. 3L, 3M, and 3N, during this movement, the legs 116 of the mobile means when moved by the operator's fingers, apply vertical downward pressure on the filter support 108 and release it from the lower end of the compartment 102. The filter and its support 108 then fall into the cell culture box or well 130.

Lastly, as illustrated in FIG. 3O, the compartment 102 and the mobile means 110 are removed from the cell culture box or well 130.

FIGS. 4-7 relate to particular embodiments of the ScreenCell® filtration device that uses a protective guiding cylinder for the vacuum tube. FIGS. 4 to 7 show the compartment 102, the movable means 110 and a protective cylinder 502 attached to the compartment 102 by a two-part connection means, 504 and 520.

The protective cylinder includes the connection means part 504, a frosted part 506, a transparent part 508 and, on an opening opposite the compartment 102, a protective film 510.

Part 520 is formed inside the end of the compartment 102. FIG. 7 shows a particular embodiment of part 520 comprising 4 prongs 522 laterally positioned on a cylindrical part in co-axial relation to the compartment 102. In this embodiment, part 504 is comprised of four grooves with profiles corresponding to those of the prongs. These grooves 524 extend in a elliptical fashion, from an opening designed to accommodate a prong 522 towards the interior of the protective cylinder 502 so that by rotating the protective cylinder 502 as indicated by an arrow in FIG. 7 causes each prong 522 to advance into the corresponding groove 524 and the protective cylinder 502 to be tightened onto the compartment 102.

The protective cylinder 502 is attached to the end-piece carrying the needle 180 as follows. The needle 180 is embedded in the lower part of part 504, which is the part facing the compartment 502. Part 504 is force-mounted onto part 506 by means of 4 spokes. The spokes are located on part 504 and are inserted into four grooves located on part 506.

Part 506 serves to isolate and protect the needle 180. The transparent part 508 enables the user to verify the status and completion of filtration.

The protective film 510, which covers and seals the entire lower opening of the cylinder 502 is furnished with a lateral part extending a short distance from the cylinder 502 (illustrated in FIG. 4). This lateral part allows the film 510 to be easily removed.

The protective film 510 protects the user from access to the needle 180. The film 510 also protects the needle 180 from the risk of clogging and/or contamination.

The cylinder 502 can be discreetly colored, for example blue, green or yellow, depending on the purposes for which the filtration device is used (cytological, molecular biology and culture studies, respectively).

Note in FIG. 5 that after the liquid to be filtered is introduced into the compartment 102 and the film 510 is removed, the vacuum tube 185 fitted with its plug 186 is inserted into the protective guiding cylinder 502. Force is then applied to the vacuum tube 185 so that the needle 180 pierces the plug 186, as explained above.

In the assemblage thus produced, shown in FIG. 6, the negative pressure initially present in the vacuum tube 185 results in filtration of the liquid present in the compartment 102.

When filtration is completed, as illustrated in FIG. 7, the protective cylinder 502 and the needle 180 it houses are jointly removed.

### Filter for Retaining Target Cells

A filter used in the methods described herein contains pores that cause the target cells to be retained on or in the filter. A suitable filter can include between 50,000 and 200,000 pores/cm² (e.g., 75,000 to 150,000 pores/cm², 90,000 to 115,000 pores/cm², or 95,000 to 110,000 pores/cm²) with an average diameter of 5.5 μm to about 7.5 μm. In one embodiment, the filter has approximately 100,000 pores/cm² with an average diameter of about 6.5 μm. The pore size used in any particular application will depend on the relative size of the target cells and non-target cells. Target cells to be retained on a filter will generally have a diameter (or longest dimension) of >20 μm and <50 μm. Naturally, at least most of the non-target cells in a fluid sample containing cells will have diameters (or largest dimensions) significantly smaller than that of the target cells in the fluid sample and smaller than the diameter (or largest dimension) of the pores in a filter of interest.

The filter can be composed of any biocompatible material, including, for example, a biocompatible polymer that is biodegradable or nonbiodegradable. Representative biocompatible polymers include, but are not limited to, poly(ester amide), polyhydroxyalkanoates (PHA), poly(3-hydroxyalkanoates) such as poly(3-hydroxypropanoate), poly(3-hydroxybutyrate), poly(3-hydroxyvalerate), poly(3-hydroxyhexanoate), poly(3-hydroxyheptanoate) and poly(3-hydroxyoctanoate), poly(4-hydroxyalkanaote) such as poly(4-hydroxybutyrate), poly(4-hydroxyvalerate), poly(4-hydroxyhexanote), poly(4-hydroxyheptanoate), poly(4-hydroxyoctanoate) and copolymers including any of the 3-hydroxyalkanoate or 4-hydroxyalkanoate monomers described herein or blends thereof, poly(D,L-lactide), poly(L-lactide), polyglycolide, poly(D,L-lactide-co-glycolide), poly(L-lactide-co-glycolide), polycaprolactone, poly(lactide-co-caprolactone), poly(glycolide-co-caprolactone), poly(dioxanone), poly(ortho esters), poly(anhydrides), poly(tyrosine carbonates) and derivatives thereof, poly(tyrosine ester) and derivatives thereof, poly(imino carbonates), poly(glycolic acid-co-trimethylene carbonate), polyphosphoester, polyphosphoester urethane, poly(amino acids), polycyanoacrylates, poly(trimethylene carbonate), poly(iminocarbonate), polyurethanes, polyphosphazenes, silicones, polyesters, polyolefins, polyisobutylene and ethylene-alphaolefin copolymers, acrylic polymers and copolymers, vinyl halide polymers and copolymers, such as polyvinyl chloride, polyvinyl ethers, such as polyvinyl methyl ether, polyvinylidene halides, such as polyvinylidene chloride, polyacrylonitrile, polyvinyl ketones, polyvinyl aromatics, such as polystyrene, polyvinyl esters, such as polyvinyl acetate, copolymers of vinyl monomers with each other and olefins, such as ethylene-methyl methacrylate copolymers, acrylonitrile-styrene copolymers, ABS resins, and ethylene-vinyl acetate copolymers, polyamides, such as Nylon 66 and polycaprolactam, alkyd resins, polycarbonates, polyoxymethylenes, polyimides, polyethers, poly(glyceryl sebacate), poly(propylene fumarate), poly(n-butyl methacrylate), poly(sec-butyl methacrylate), poly(isobutyl methacrylate), poly(tert-butyl methacrylate), poly(n-propyl methacrylate), poly(isopropyl methacrylate), poly(ethyl methacrylate), poly(methyl methacrylate), epoxy resins, polyurethanes, rayon, rayon-triacetate, cellulose acetate, cellulose butyrate, cellulose acetate butyrate, cellophane, cellulose nitrate, cellulose propionate, cellulose ethers, carboxymethyl cellulose, copoly(ether-esters) (e.g., poly(ethylene oxide/poly(lactic acid) (PEO/PLA)), polyalkylene oxides such as poly(ethylene oxide), poly(propylene oxide), poly(ether ester), polyalkylene oxalates, polyphosphazenes, phosphoryl choline, choline, poly(aspirin), polymers and co-polymers of hydroxyl bearing monomers such as 2-hydroxyethyl methacrylate (HEMA), hydroxypropyl methacrylate (HPMA), or hydroxypropylmethacrylamide, carboxylic acid bearing monomers such as methacrylic acid (MA), acrylic acid (AA), alkoxymethacrylate, alkoxyacrylate, or 3-trimethylsilylpropyl methacrylate (TMSPMA). See for example, U.S. Pat. No. 7,887,572. Track-etched filters composed of one or more of such polymers are particularly useful as the track-etched manufacturing process results in a more precise pore size with a narrow pore size distribution. In one embodiment, the filter is a polycarbonate filter. In one embodiment, the filter is a polycarbonate track-edged filter from Whatman (Kent, UK), EMD Millipore (Billerica, Mass.), Membrane solutions (Plano, Tex.), or it4ip (Seneffe, Belgium). Polycarbonate track-edged filters can be implanted in an immunosuppressed mouse with no adverse effects.

In some embodiments, the surface of the filter (e.g., a polycarbonate filter) can be modified by, for example, immobilization of one or more compounds or by treatment to render the surface more hydrophilic. For example, one or more of a growth factor, an extracellular matrix protein, an enzyme, a reporter molecule, a liposome, or a nucleic acid can be immobilized on the filter. Non-limiting examples of growth factors include epidermal growth factor (EGF), platelet derived growth factor (PDGF), keratinocyte growth factor (KGF), a fibroblast growth factor (FGF), and a transforming growth factor (TGF). Non-limiting examples of extracellular matrix proteins include collagen, laminin, fibronectin, and heparan sulfate.

In one embodiment, one or more reporter molecules can be immobilized on the filter such that cell growth can be detected during culture of the cells or after implantation in an immunodeficient non-human animal. For example, when the target cell is a tumor cell, a reporter molecule can include a fluorophore-quencher dual labeled probe that is a substrate for a metalloproteinase (MMP). For example, a substrate such as MCA-Pro-Leu-Gly-Leu-DPA-Ala-Arg-NH, where MCA refers to methoxycoumarin and DPA refers to dinitrophenyl, can be used. Most MMPs associated with tumor growth can cleave such as substrate. Fluorescent MCA is quenched by DPA until the peptide is cleaved by a MMP between the Gly and Leu residues. Detecting of fluorescent MCA is indicative of growth of the cells. One of skill in the art will appreciate that other combinations of fluorophore and quencher molecules can be used.

In one embodiment, the reporter molecule immobilized on the filter is an in vivo targeted, activatable optical imaging probe based on a fluorophore-quencher pair bound to a targeting ligand. See, Ogawa et al., Mol. Pharm. 6(2): 386-395 (2009). With this system, fluorescence is quenched by the fluorophore-quencher interaction outside the target cells, but is activated within the target cells by dissociation of the fluorophore-quencher pair in lysosomes/endosomes. The rhodamine core fluorophore TAMRA and QSY7 quencher pair are particularly useful for in vivo imaging. Suitable target ligands include for example, a receptor ligand such as avidin, which is a noncovalently bound homotetrameric glycoprotein that binds to D-galactose receptor. D-galactose receptor is expressed on many cancer cells including ovarian, colon, gastric, and pancreatic cancer cells. A targeting ligand also can be an antibody or antigen-binding fragment thereof that has binding affinity for a tumor specific antigen such as human epidermal growth factor receptor type 2 (HER2) expressed on the cell surface of some tumors. See, Ogawa et al., 2009, supra.

In some embodiments, an antibody or antigen-binding fragment thereof can be immobilized on a filter. Such an antibody or antigen-binding fragment thereof can be immobilized on the filter before or after filtering the sample. It will be appreciated that immobilization of the antibody or fragment thereof on the filter, however, would not substantially contribute to the selection of target cells during the filtering process. Such an antibody or fragment thereof, however, can be useful for acting as a growth promoting ligand during the culture of the cells or after implantation in an immunodeficient non-human animal.

“Antibody” as the term is used herein refers to a protein that generally comprises heavy chain polypeptides and light chain polypeptides. Antigen recognition and binding occurs within the variable regions of the heavy and light chains. Single domain antibodies having one heavy chain and one light chain and heavy chain antibodies devoid of light chains are also known. A given antibody comprises one of five types of heavy chains, called alpha, delta, epsilon, gamma and mu, the categorization of which is based on the amino acid sequence of the heavy chain constant region. These different types of heavy chains give rise to five classes of antibodies, IgA (including IgA1 and IgA2), IgD, IgE, IgG (IgG1, IgG2, IgG3 and IgG4) and IgM, respectively. A given antibody also comprises one of two types of light chains, called kappa or lambda, the categorization of which is based on the amino acid sequence of the light chain constant domains. IgG, IgD, and IgE antibodies generally contain two identical heavy chains and two identical light chains and two antigen combining domains, each composed of a heavy chain variable region (V_{H}) and a light chain variable region (V_{L}). Generally IgA antibodies are composed of two monomers, each monomer composed of two heavy chains and two light chains (as for IgG, IgD, and IgE antibodies); in this way the IgA molecule has four antigen binding domains, each again composed of a V_{H} and a V_{L}. Certain IgA antibodies are monomeric in that they are composed of two heavy chains and two light chains. Secreted IgM antibodies are generally composed of five monomers, each monomer composed of two heavy chains and two light chains (as for IgG and IgE antibodies); in this way the IgM molecule has ten antigen binding domains, each again composed of a V_{H} and a V_{L}. A cell surface form of IgM also exists and this has two heavy chain/two light chain structure similar to IgG, IgD, and IgE antibodies.

“Antigen binding fragment” of an antibody as the term is used herein refers to an antigen binding molecule that is not a complete antibody as defined above, but that still retains at least one antigen binding site. Antibody fragments often include a cleaved portion of a whole antibody, although the term is not limited to such cleaved fragments. Antigen binding fragments can include, for example, a Fab, F(ab′)₂, Fv, and single chain Fv (scFv) fragment. An scFv fragment is a single polypeptide chain that includes both the heavy and light chain variable regions of the antibody from which the scFv is derived. Other suitable antibodies or antigen binding fragments include linear antibodies, multispecific antibody fragments such as bispecific, trispecific, and multispecific antibodies (e.g., diabodies (Poljak Structure 2(12):1121-1123 (1994); Hudson et al., J. Immunol. Methods 23(1-2):177-189 (1994)), triabodies, tetrabodies), minibodies, chelating recombinant antibodies, intrabodies (Huston et al., Hum. Antibodies 10(3-4):127-142 (2001); Wheeler et al., Mol. Ther. 8(3):355-366 (2003); Stocks Drug Discov. Today 9(22): 960-966 (2004)), nanobodies, small modular immunopharmaceuticals (SMIP), binding-domain immunoglobulin fusion proteins, camelid antibodies, camelized antibodies, and V_{HH} containing antibodies.

Non-limiting examples of antibodies or antigen-binding fragments thereof that act as growth promoting ligands include anti-CD3 antibodies for T cell tumors; anti-Ig antibodies for B cell tumors; or antibodies that can induce dimerization of class 1 growth factor receptors. See, for example, Fuh, et al. (1992) Science 256: 1677-1680; Rui, et al. (1994) Endocrinology 135: 1299-1306; Schneider, et al. (1997) Blood 89: 473-482; Mahanta, et al. (2008) PLoS One. 3(4):e2054; and Spaargaren, et al. (1991) J Biol Chem. 266(3):1733-9.

### Implanting Filters in Immunodeficient Non-Human Animals

After recovering one or more target cells on the filter, the filter can be implanted in a non-human animal, most commonly an immunodeficient non-human animal (e.g., an immunodeficient rodent such as an immunodeficient mouse or rat). The immunodeficient non-human animal can be homozygous for the severe combined immune deficiency (SCID) spontaneous mutation (Prkdc^{scid}); homozygous for the nude spontaneous mutation (Foxn1^{nu/nu}); homozygous for a Rag1 mutation; homozygous for a Rag2 mutation; or homozygous for both the Rag1 and the Rag2 mutations. Such immunodeficient non-human animals (e.g., immunodeficient mice) are commercially available from, for example, The Jackson Laboratory (Bar Harbor, Me.). Typically, the filter is surgically implanted into the immunodeficient animal subcutaneously. For example, the filter can be implanted under the neural crest, under the adrenal gland capsule, in the peritoneal cavity, or flank of the animal. In some embodiments, a compound such as a growth factor or reconstituted basement membrane matrix can be administered to the animal before, during, or after the filter is implanted.

In some embodiments, one to four additional filters are implanted in the non-human animal, where each filter contains one or more target cells. For example, one, two, three, or four filters can be implanted in the non-human animal. Each filter can be obtained from a single filtration device or can be obtained from separate filtration devices. Typically, when multiple filters are implanted into one animal, all of the filters contain cells recovered from the same patient. The filters can be implanted in different regions of the animal, e.g., in each flank or flank and abdomen of the animal.

In some embodiments, before implantation, the surface of the filter can be contacted with a composition that can transition from a liquid to gel phase without lethal or toxic effects on the target cells, for example, without the use of chemicals or temperatures that would harm living cells, e.g., kill or inhibit the proliferative capacity of the cells. In addition, the constituents of the compositions should not be toxic or lethal to cells or anti-proliferative. For example, the composition can be a hydrogel composed of crosslinked polymer chains, natural or synthetic in origin, such as Puramatrix™ (a synthetic peptide matrix) from 3DM, Inc (Cambridge, Mass.), or the polyethylene (glycol) diacrylate-based, hyaluron based, or collagen based hydrogels from Glycosan BioSystems (Salt Lake City, Utah). Such hydrogels can be applied in liquid form to the filter and then transitioned to the gel phase by adding culture medium. Filters also can be contacted with Matrigel™ (BD Biosciences) reconstituted basement membrane matrix or a composition containing Matrigel™ and a culture medium. The composition also can contain one or more extracellular matrix components, e.g., proteoglycans (such as heparan sulfate, chondroitin sulfate, and keratan sulfate), hyaluronic acid, collagen type IV, elastin, fibronectin, and laminin. Growth factors or other molecules can be added to the compositions as needed for culture of the target cells.

In embodiments in which two or more filters are implanted, the filters can be stacked substantially on top of each other to produce a multi-layered three-dimensional culture device. Before stacking the filters, the surface of the filters can be contacted with the above-described composition that can transition from a liquid to gel phase without lethal or toxic effects on the target cells. For example, the surface of the filter can be contacted with a reconstituted basement membrane matrix.

In some embodiments, before implanting, the one or more filters can be placed in a cell culturing device and cultured in the presence of a culture medium to, for example, assess viability or increase cell number. In some embodiments, once cell number of the target cells has increased, the target cells can be removed from the filter (e.g., by washing) and implanted in the immunodeficient animal.

After implanting the one or more filters in the immunodeficient non-human animal, the animal can be monitored for growth of the cells or development of a tumor. For example, to monitor growth of cells, the implanted filter can include a substrate for a MMP or an in vivo activatable optical imaging probe as discussed above. Development of a tumor in the animals confirms the presence of tumor cells in the fluid sample and is indicative of the metastasis potential of the cells. A tumor can be removed from an animal and subjected to further in vitro or in vivo characterization. For example, a tumor or cells isolated from the tumor can be subjected to genomic, proteomic, immunocytochemical, or other molecular assays to further characterize the tumor and/or cells. In some embodiments, tissue specific and/or tumor specific reagents such as antibodies, probes, or PCR primers can be used to examine a tumor or cells isolated from the tumor.

In embodiments in which the growth of tumor cells has been confirmed in the animal model, responsiveness of the cells to one or more chemotherapeutic agents can be assessed by administering the chemotherapeutic of interest to the animal and monitoring responsiveness (e.g., by monitoring cell growth or cell death).

The invention is further described in the following examples, which do not limit the scope of the invention described in the claims.

### EXAMPLES

### Example 1

### Use of the ScreenCell® Filtration Device

This non-limiting example provides the general method of use of a filtration device from ScreenCell® (ScreenCell® cell culture (CC)). The device is 19 cm long and includes a circular track-etched polycarbonate filter (e.g., from Whatman, EMD Millipore, Membrane Solution, or it4ip) with a smooth, flat and hydrophilic surface.

The filter contains circular pores having a diameter of 6.5 μm, randomly distributed throughout the filter (1×10⁵ pores/cm²). According to the manufacturer of the device, before filtration and in order to lyse red blood cells (RBCs), 3 to 6 ml blood samples are diluted in 3 to 1 ml of ScreenCell® LC buffer, respectively. That is, a 3 ml blood sample is diluted with 3 ml of buffer while a 6 ml blood sample is diluted with 1 ml of buffer. After mixing the sample and dilution buffer and incubating for 2 minutes at room temperature, 2.6 or 1.6 ml of culture medium are added, respectively, for a total volume of 8.6 ml, then the sample is passed through the filtration device to which a vacuum tube is attached. Filtration is usually complete within approximately 2 minutes. At the end of filtration, the nozzle/holder of the ScreenCell® device is unclipped and removed from the filtration tank and the filter is released into a well of a 24-well tissue culture plate, by pushing down evenly a rod located at the bottom part of the filtration device. Adequate tissue culture medium and growth factors can be added into the well. The filtration area of the ScreenCell® CC device filter is delimited by an O ring made of surgical inox with a numeric code to insure traceability of the filtered samples.

In order to increase the number of rare cells obtained from a sample, cells can be recovered from multiple portions of the same diluted blood sample. Each portion can be filtered through a different filtration device. When multiple filters are obtained, the filters can be laid successively upon each other as described above. For example, a filter can be released from a filtration device and placed on a 100 μl layer of 1:1 Matrigel/Medium (M/M). Each successive filter is covered with 70 μl of M/M before laying down the next filter. At the end of the process, the last filter is covered with 70 μl of M/M, and a sufficient volume of culture medium (e.g., culture medium containing fetal calf serum (FCS)) is added to cover the stack of filters to provide a three-dimensional culture device. The three-dimensional device can be used to transport and/or culture the cells, and also can be implanted in an immunodeficient animal.

### Example 2

### Sensitivity of the ScreenCell® Device for Detecting CTCs

The sensitivity of the ScreenCell® device (described in Example 1) for isolating CTCs was assessed as follows. Twenty five independent experiments were conducted with fixed H2030 cells (an adenocarcinoma non-small cell lung cancer cell line from the American Type Culture Collection (ATCC); Catalog No. CRL-5914™). The H2030 cells were cultured in flasks containing RPMI 1640 supplemented with 10% FCS and harvested by trypsinization. Cell viability was assessed by trypan blue exclusion. The cells were used in the experiments described below if viability was estimated to exceed 90%.

After fixation with formaldehyde, the H2030 cells were spiked into whole peripheral blood drawn from a healthy donor to yield a final concentration of 2 or 5 fixed H2030 cells per 1 mL of blood, and filtered through the ScreenCell® device as set forth in Example 1. The average filtration time was 50 seconds. Cells on the filters were stained with hematoxylin and eosin, and counted. The number of H2030 cells spiked into the blood sample versus the actual number of H2030 cells recovered in the sample is shown in Tables 1 and 2. For the samples spiked with 5 cells, the average percentage of H2030 cells recovered was 91.2%, with an average of 4.56±0.71 cells recovered. See Table 1. In the samples spiked with 5 cells, no fewer than 3 cells were detected in all 25 samples. For the samples spiked with 2 cells, the average percentage of H2030 cells recovered was 74% with an average of 1.480±0.71 cells recovered. See Table 2.

TABLE 1

| | | Exp. | Exp. | Exp. | Exp. | Exp. | |
|---|---|---|---|---|---|---|---|
| | | #1 | #2 | #3 | #4 | #5 | Total |
| Number of cells | | 5 | 5 | 5 | 5 | 5 | 25 |
| spiked in 1 | | | | | | | |
| mL of blood | | | | | | | |
| per filter | | | | | | | |
| Number, | Filter | 4, 80 | 5, 100 | 4, 80 | 5, 100 | 4, 80 | 22 |
| Percent of | #1 | | | | | | |
| Cells | Filter | 5, 100 | 5, 100 | 5, 100 | 3, 60 | 5, 100 | 23 |
| Recovered | #2 | | | | | | |
| per filter | Filter | 3, 60 | 4, 80 | 5, 100 | 5, 100 | 5, 100 | 22 |
| | #3 | | | | | | |
| | Filter | 5, 100 | 4, 80 | 4, 80 | 5, 100 | 5, 100 | 23 |
| | #4 | | | | | | |
| | Filter | 4, 80 | 5, 100 | 5, 100 | 5, 100 | 5, 100 | 24 |
| | #5 | | | | | | |

| | | | | | | |
|---|---|---|---|---|---|---|
| Total Number of | 25 | 23 | 25 | 25 | 25 | 125 |
| Spiked Cells | | | | | | |
| Total Number | 21 | 23 | 23 | 23 | 24 | 114 |
| of cells | | | | | | |
| isolated on | | | | | | |
| the 5 filters | | | | | | |
| Average % | 84% | 92% | 92% | 92% | 96% | 91.2 |
| recovery | | | | | | |
| per filter | | | | | | |

TABLE 2

| | | Exp. | Exp. | Exp. | Exp. | Exp. | |
|---|---|---|---|---|---|---|---|
| | | #1 | #2 | #3 | #4 | #5 | Total |
| Number of cells | | 2 | 2 | 2 | 2 | 2 | 10 |
| spiked in 1 | | | | | | | |
| mL of blood | | | | | | | |
| per filter | | | | | | | |
| Number, | Filter | 2, 100 | 1, 50 | 2, 100 | 1, 50 | 2, 100 | 8 |
| Percent of | #1 | | | | | | |
| Cells | Filter | 2, 100 | 1, 50 | 0, 0 | 2, 100 | 2, 100 | 7 |
| Recovered | #2 | | | | | | |
| per filter | Filter | 0, 0 | 2, 100 | 2, 100 | 1, 50 | 2, 100 | 7 |
| | #3 | | | | | | |
| | Filter | 2, 100 | 2, 100 | 1, 50 | 0, 0 | 2, 100 | 7 |
| | #4 | | | | | | |
| | Filter | 2, 100 | 1, 50 | 2, 100 | 1, 50 | 2, 100 | 8 |
| | #5 | | | | | | |

| | | | | | | |
|---|---|---|---|---|---|---|
| Total Number of | 10 | 10 | 10 | 10 | 10 | 50 |
| Spiked Cells | | | | | | |
| Total Number | 8 | 7 | 7 | 5 | 10 | 37 |
| of cells | | | | | | |
| isolated on | | | | | | |
| the 5 filters | | | | | | |
| Average % | 80 | 70 | 70 | 50 | 100 | 74 |
| recovery per | | | | | | |
| filter | | | | | | |

To verify whether the percentage of cell loss was related to the filtration device, H2030 cells were harvested as indicated above, fixed, and pipetted directly into an Eppendorf tube containing filtration buffer. Cells were recovered using a Cytospin centrifuge and stained with hematoxylin and eosin. Under these conditions, the mean percentage of recovery was 82% for samples spiked with 2 cells and 88% for samples spiked with 5 cells. For the samples spiked with 2 cells, an average of 1.64±0.57 cells was recovered. For the samples spiked with 5 cells, an average of 4.40±0.71 cells was recovered. The relative sensitivities of the ScreenCell® device versus direct cell collection were assessed through P-values calculated for unpaired unilateral Student test (0.19 and 0.20 for 2 and 5 spiked cells, respectively), unpaired bilateral Student test (0.39 and 0.41 for 2 and 5 cells respectively), and Fisher test (0.14 and 0.34 for 2 and 5 cells respectively). These tests showed that collection of 2 or 5 spiked tumor cells through the Screencell® device or by direct collection of the micropipetted cells directly into an Eppendorf tube resulted in similar sensitivities. Through the different series of tests using the Screencell® device and direct collection, similar numbers of cells were lost after 25 independent collections of 2 or 5 spiked tumor cells. Indeed, the percentage of cells lost through the Screencell® device was 26% (standard deviation (SD) was 0.71 with an average number of cells lost of 0.52), and 9% (SD was 0.65 with an average number of cell lost of 0.44) for 2 and 5 spiked H2030 cells respectively, while it was 18% (SD was 0.57 with an average number of cell lost of 0.36) and 12% (SD was 0.71 with an average number of cell lost of 0.60) through direct collection. The P-value for unpaired unilateral Student test indicated similar numbers of lost cells using ScreenCell® Cyto device or by direct collection with 2 or 5 tumor cells.

No significant differences were found when using the P-value for the unpaired unilateral Student test to compare the results obtained with 2 versus 5 spiked tumor cells through the ScreenCell® device or by direct collection (0.19 versus 0.20 for 2 and 5 spiked tumor cells, respectively). Furthermore, no significant differences were found when using the P-value for the unpaired unilateral Student test to compare the results obtained with 2 versus 5 spiked tumor cells through the ScreenCell® device (0.34) or by direct collection (0.10). Altogether, these results indicate that cells were lost essentially through micropipetting and that the recovery rate of the ScreenCell® device was close to 100%.

### Example 3

### Viability and Culture of Cells Following Filtration Through a ScreenCell® Device

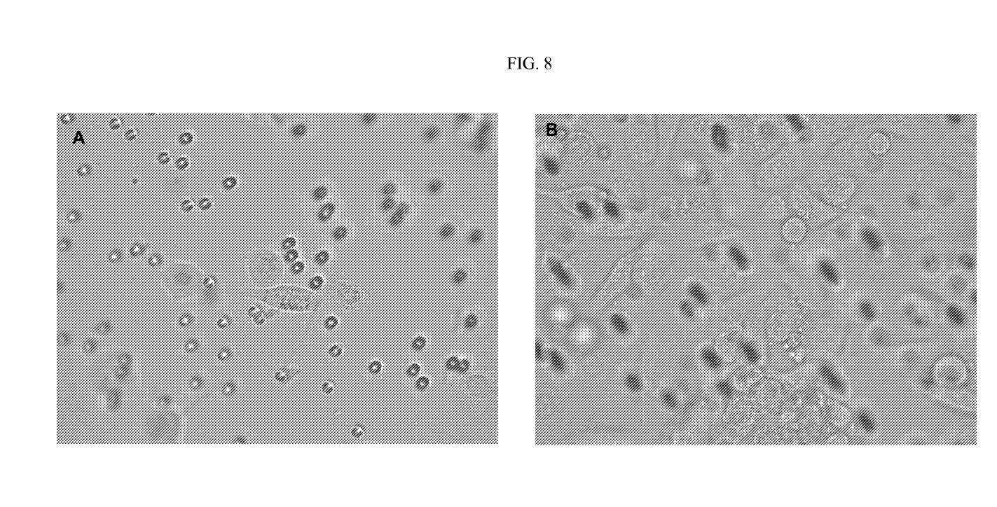

Five independent experiments were conducted to assess the viability of tumor cells after filtration through the ScreenCell® CC device of Example 1. H2030 cells were cultured in flasks containing RPMI 1640 supplemented with 10% FCS and harvested by trypsinization. Fifty (50) live H2030 cells were filtrated through the filtration device as described in Example 1. Viable cells were counted immediately after filtration using the trypan blue exclusion test. FIG. 8A is a photomicrograph of live H2030 cells following filtration through a ScreenCell® Cyto device. The mean percent recovery was 85%±9%. The capacity of isolated H2030 cells to grow in tissue culture was further evaluated in eight independent experiments. In each experiment, isolated H2030 cells were able to grow and expand on the filter under adequate tissue culture conditions. FIG. 8B is a photomicrograph of the cells from FIG. 8A after culturing of the filter for 4 days in culture medium.

### Example 4

### Recovery of Target Cells on a Filter and Transfer of the Filter to an Immunodeficient Animal

To demonstrate that CTCs can be transferred from a human patient to a mouse, human HT29 colorectal cancer cells were grown to 50% confluence and trypsinized to release the cells from the surface of the culture dish. The cells were washed in PBS and diluted to yield a final concentration of 10,000 cells per μ. Normal human blood was collected in a sterile EDTA Vacutainer and used within 60 minutes of collection. Either 1000 or 10,000 HT29 cells were added to 6 ml of normal human blood and the blood was gently mixed. Six ml of blood containing the HT29 cells then was diluted with 1 ml of ScreenCell® LC dilution buffer and incubated for 2 minutes at room temperature. After 2 minutes incubation, 1.6 ml of culture medium were added and the whole 8.6 ml of diluted blood then was passed through the ScreenCell® CC device described in Example 1. Upon completion of filtration, the filter was ejected onto a piece of sterile gauze. Sterile forceps were used to place the filter on a solidified pad containing 100 μl of a 1:1 ratio of Matrigel/Medium. This solidified M/M pad was used to keep cells hydrated during the surgical procedure.

The filter was implanted under the skin of a Rag2^{-/-} immunodeficient mouse and cell growth was monitored by palpation. A tumor developed on both sides of the ScreenCell® filter within 3 weeks. The mouse was sacrificed and the filter with attached tumor was removed and placed in a Petri dish. Cells from the tumor were cultured and stained with antibodies, confirming that the tumor was derived from HT29 cells. Herrmann et al. (PLoS ONE, 2010, 5:1-10) show that CD44^{high}/CD24^{high}/EpCAM^{high} HT29 cells selected in vivo had a cancer stem cell phenotype.

### OTHER EMBODIMENTS

While the invention has been described in conjunction with the foregoing detailed description and examples, the foregoing description and examples are intended to illustrate and not to limit the scope of the invention, which is defined by the scope of the appended claims. Other aspects, advantages, and modifications are within the scope of the claims.

1. A method of increasing the number of target cells from a fluid sample comprising cells, said method comprising:
a) providing a filter comprising one or more target cells, said one or more target cells obtained from a fluid, target cell and non-target cell containing sample by passage of the sample through a filtration device comprising said filter, wherein the size of pores in the filter causes the target cells to be retained on or in said filter; and
b) implanting said filter and said one or more target cells on said filter in an immunodeficient non-human animal, wherein some or all of the one or more cells on or in the implanted filter proliferate in said immunodeficient animal.

2. The method of claim 1, wherein, during the passage of the sample through said filtration device, substantially all the non-target cells pass through said filter.

3. The method of claim 1, wherein said immunodeficient non-human animal is a mouse.

4. The method of claim 3, wherein said mouse is homozygous for the severe combined immune deficiency (SCID) spontaneous mutation (Prkdc^{scid}); homozygous for the nude spontaneous mutation (Foxn1^{nu/nu}); homozygous for a Rag1 mutation; homozygous for a Rag2 mutation; or homozygous for both the Rag1 and the Rag2 mutation.

5. The method of claim 1, said method comprising providing one to four additional filters, each said additional filter comprising one or more target cells, and implanting said first and additional filters in said immunodeficient non-human animal.

6. The method of claim 5, wherein the one to four additional filters are one additional filter or to additional filters.

7. (canceled)

8. The method of claim 5, wherein the first and additional filters were obtained from a single filtration device or were each obtained from a separate filtration device.

9. (canceled)

10. The method of claim 5, said method further comprising, before implanting said filter and said one or more target cells on said filter, stacking said filters substantially on top of each other to produce a multi-layered culture device.

11. The method of claim 1, said method further comprising, before implanting said filter and said one or more target cells on said filter, contacting the surface of said filter and any additional filters comprising said target cells with a composition that can transition from a liquid to gel phase without lethal or toxic effects on the target cells.

12. The method of claim 11, wherein said composition comprises one or more extracellular matrix (ECM) components.

13. The method of claim 12, wherein the composition comprises reconstituted basement membrane.

14. (canceled)

15. The method of claim 5, wherein said filter or the additional filters comprise one or more compounds immobilized thereto.

16. The method of claim 1, wherein one or more compounds are administered to the immunodeficient animal.

17. The method of claim 16, wherein said one or more compounds are selected from the group consisting of a growth factor, an extracellular matrix protein, an enzyme, a reporter molecule, a liposome, and a nucleic acid.

18. The method of claim 17, wherein said growth factor is epidermal growth factor (EGF), platelet derived growth factor (PDGF), keratinocyte growth factor (KGF), a fibroblast growth factor (FGF), or a transforming growth factor (TGF); wherein said extracellular matrix protein is collagen, laminin, fibronectin, or heparan sulfate, or wherein said reporter molecule comprises a fluorophore-quencher dual labeled probe that is a substrate for a metalloproteinase.

19. (canceled)

20. (canceled)

21. The method of claim 1, said method further comprising monitoring growth of said cells in said immunodeficient animal.

22. The method of claim 1, wherein said fluid, cell-containing sample comprises peripheral blood cells, cells from urine, bone marrow, lymph, lymph node, spleen, cerebral spinal fluid, ductal fluid, a biopsy specimen, or a needle biopsy aspirate.

23. (canceled)

24. The method of claim 1, said method further comprising, before said implanting step, culturing said one or more target cells.

25. (canceled)

26. The method of claim 1, wherein said target cells are cancer cells, circulating cancer cells, fetal cells, stem cells, endothelial stem cells, or mesenchymal stem cells.

27-29. (canceled)

30. A non-human immunodeficient animal comprising at least one implanted filter, said filter comprising a plurality of target cells obtained from a fluid, target cell and non-target cell containing sample by passage through a filtration device comprising said filter, wherein the size of pores in the filter causes the target cells to be retained on or in said filter.

31. The animal of claim 30, said animal further comprising one to four additional implanted filters, each said additional filter comprising one or more target cells.

32. (canceled)

33. (canceled)

34. The animal of claim 31, wherein the first and additional filters were obtained from a single filtration device or were obtained from a separate filtration device.

35. (canceled)

36. The animal of claim 31, wherein the first and additional filters are substantially stacked on top of each other to produce a multi-layered three-dimensional culture device.

37. The animal of claim 30, wherein the surface of said filter and any additional filters comprising said target cells contains a composition that can transition from a liquid to gel phase without lethal or toxic effects on the target cells.

38. The animal of claim 30, wherein said target cells are human target cells.

39. A method of testing for the presence of tumor cells in fluid sample comprising test cells, said method comprising:
a) providing a filter comprising a plurality of test cells obtained from a fluid, test cell-containing sample by passage through a filtration device comprising said filter, wherein the size of pores in the filter causes one or more of the test cells to be retained on or in said filter;
b) implanting said filter and said one or more of the test cells on or in said filter in said immunodeficient non-human animal; and
c) monitoring said immunodeficient non-human animal for the presence or absence of a tumor, wherein the presence of a tumor indicates that the test cells comprised tumor cells.

40. The method of claim 39, wherein said test cells are human cells.

41. The method of claim 40, wherein said fluid, cell-containing sample comprises peripheral blood cells, cells from urine, bone marrow, lymph, lymph node, spleen, cerebral spinal fluid, ductal fluid, a biopsy specimen, or a needle biopsy aspirate.

42. (canceled)

43. The method of claim 39, said method further comprising d) administering a chemotherapeutic agent to said non-human animal if said tumor is present; and e) monitoring said tumor for responsiveness to said chemotherapeutic agent.

44. A method of increasing the number of target cells from a fluid sample comprising cells, said method comprising:
a) providing a fluid, target cell- and non-target cell-containing sample;
b) passing said sample through a filtration device, said device comprising a filter support fastened to a filter, a compartment having an upper opening and a lower opening, and means mobile relative to said compartment for applying a force to the support and releasing said support;
c) removing, from said device, said filter containing one or more target cells; and
d) implanting said filter and said target cells on or in said filter into said immunodeficient non-human animal, wherein some or all of the one or more target cells on or in the implanted filter proliferate in said immunodeficient animal.
